(19) United States
(12) Patent Application Publication
(10) Pub. No.: US 2017/0157296 A1
(43) Pub. Date: Jun. 8, 2017

(54) MACROPHAGE OR MONOCYTE ENHANCED WOUND HEALING

(71) Applicant: The Board of Trustees of the Leland Stanford Junior University, Stanford, CA (US)

(72) Inventors: Graham G. Walmsley, Menlo Park, CA (US); Kipp Andrew Weiskopf, Sudbury, MA (US); Michael Sung-Min Hu, Palo Alto, CA (US); Michael T. Longaker, Atherton, CA (US); Irving L. Weissman, Stanford, CA (US); Geoffrey C. Gurtner, Woodside, CA (US); Jayakumar Rajadas, Cupertino, CA (US)

(21) Appl. No.: 15/327,324

(22) PCT Filed: Jul. 20, 2015
(86) PCT No.: PCT/US15/41155

§ 371 Date: Jan. 18, 2017

Related U.S. Application Data
(60) Provisional application No. 62/026,853, filed on Jul. 21, 2014.

(51) Int. Cl. A61L 27/60; A61L 27/26; A61L 27/56; A61L 27/52

(52) U.S. Cl. CPC A61L 27/60; A61L 27/52; A61L 27/26; A61L 27/56; A61L 2300/30

(57) ABSTRACT

Compositions and methods are provided for enhanced healing of wounds, e.g. cutaneous wounds, by application of a scaffold or matrix, e.g. a hydrogel film comprising a dose of macrophages, or monocyte progenitors thereof, which dose is effective in increasing the rate of wound healing. The compositions of the invention find use in treating cutaneous wounds, particularly chronic wounds, e.g. in diabetic patients or other patients with impaired wound healing.

### CROSS REFERENCE

This application is a 371 application and claims the benefit of PCT Application No. PCT/US2015/041155, filed Jul. 20, 2015, which claims benefit of U.S. Provisional Patent Application No. 62/026,853, filed Jul. 21, 2014, which applications are incorporated herein by reference in their entirety.

### BACKGROUND

Normal wound healing is the interaction of a complex cascade of cellular events that generates resurfacing, reconstitution, and restoration of the tensile strength of injured skin, traditionally explained in terms of 4 overlapping classic phases: hemostasis, inflammation, proliferation, and maturation.

In some instances, wound healing does not proceed normally. For example, diabetes has multiple effects on wound healing. Microangiopathic disease results in impaired tissue blood flow and subsequent poor tissue oxygen delivery. Peripheral neuropathy predisposes to tissue trauma and an increased risk for infection. In addition, decreased immune function, slower collagen synthesis and accumulation, decreased angiogenesis, and poorer tensile strength of wounds leads to a high risk of wound dehiscence. Adequate control of blood glucose levels plays a crucial role in the diabetic healing wound. Although definite parameters have not been defined, glucose levels of greater than 200 mg/dL are associated with worse outcomes.

Smoking also has a known negative effect on healing due to a direct toxic effect and the vasoconstriction induced by nicotine. Smoking has also been shown to decrease the function of neutrophils, inhibit collagen synthesis, and increase levels of carboxy-hemoglobin, owing to the effects of carbon monoxide and hydrogen cyanide. Other factors contributing to poor wound healing include nutritional deficiency, prior radiation therapy, active wound infection, and tissue hypoxia.

Tissue engineering of skin requires biomaterial techniques capable of recapitulating both cellular and non-cellular elements. An important non-cellular element that plays a critical role in regulating skin behavior is the dermal extracellular matrix (ECM). This complex environment not only houses the myriad cell types involved in skin homeostasis and repair, but also provides mechanical stability, enables metabolite and cellular movement, and is constantly remodeled in response to local and systemic cues. Dermal scaffolds, derived from both native and synthetic sources, constitute the foundation for skin replacement techniques and have been used with variable success. Native dermal sources, such as decellularized cadaveric skin, are limited by cost, donor availability, and disease transmission concerns.

Improved skin substitutes that provide for improved healing are desirable for many clinical purposes.

### PUBLICATIONS

United States Patent Application 20110305745, entitled “Pullulan regenerative matrix”, Dec. 15, 2011. Wong et al. Tissue Engineering Part A. March 2011, 17(5-6): 631-644, entitled “Engineered Pullulan—Collagen Composite Dermal Hydrogels Improve Early Cutaneous Wound Healing”; Wong et al. (2011), Macromol. Biosci., 11:1458-1466, entitled “Pullulan Hydrogels Improve Mesenchymal Stem Cell Delivery into High-Oxidative-Stress Wounds”. Wang et al. (2012). J Biomed Mater Res Part A 2012:100A:1438-1447, entitled “A cellular delivery system fabricated with autologous BMSCs and collagen scaffold enhances angiogenesis and perfusion in ischemic hind limb”.

Franzen et al. (1998) J. Neuroscience res. 51(3):316-327, entitled “Effects of macrophage transplantation in the injured adult rat spinal cord: A combined immunocytochemical and biochemical study”. Francke et al. American Journal of Translational Research 2013; 5(2):155-169, entitled “Transplantation of bone marrow derived monocytes: a novel approach for augmentation of arteriogenesis in a murine model of femoral artery ligation.”

### SUMMARY OF THE INVENTION

Compositions and methods are provided for enhanced healing of wounds, e.g. cutaneous wounds, by application of a scaffold or matrix, e.g. a hydrogel, comprising a dose of macrophages, or monocyte progenitors thereof, which dose is effective in increasing the rate of wound healing. The compositions of the invention find use in treating cutaneous wounds, particularly chronic wounds, e.g. in diabetic patients or other patients with impaired wound healing. The dose of macrophages or monocytes is sufficient to enhance the rate of healing. The cells can be autologous or allogeneic with the recipient.

In the methods of the invention, a wound, e.g. a chronic cutaneous wound, is contacted at the surface with a scaffold or matrix, e.g. a hydrogel comprising macrophages or monocyte progenitors thereof. The wound dressing may include a support or covering over the scaffold or matrix as a barrier. The dressing can remain in place until the wound is healed, e.g. for up to about 3, 5, 7, 9, 11, 13, 15, 17 or more days. Alternatively the dressing can be changed after a suitable period of time, e.g. after about 3, 5, 7, 9, 11, 13, 15, 17 or more days.

In some embodiments of the invention, a hydrogel-cell composition is provided, which hydrogel is comprised of a cross-linked pullulan scaffold, for example a hydrogel comprising from about 5% to about 40% pullulan by weight when hydrated; and collagen at a concentration of from about 1% to about 10% of the total dry weight of the hydrogel, excluding cells. The hydrogel may comprise pores of controlled size, usually pores of from about 10-100 μm in diameter. Present in the hydrogel is an effective dose of macrophages or monocyte progenitors thereof within the scaffold, where the effective dose may be from about 10⁴ cells/cm² of hydrogel, up to about 10⁸ cells/cm² of hydrogel, and may be present at a concentration of from about 10⁵ to about 10⁷ cells/cm² of hydrogel, assuming that the hydrogel is a planar configuration of from about 0.5 to about 2.5 mm in thickness. The cells are typically enriched for macrophages or monocyte progenitors thereof, for example by selection for expression of CD14, for adherence, etc., and the cell population may be at least about 50% the desired cell type, e.g. at least about 50% CD14⁺; at least about 60% the desired cell type; at least about 70% the desired cell type; at least about 80% the desired cell type; at least about 90% the desired cell type; at least about 95% the desired cell type.

Optionally the hydrogel is fabricated with salt-induced phase inversion and cross-linking to form a reticular scaffold. This soft pullulan-collagen scaffold displays excellent handling characteristics, durability, and a porous dermal-like ultrastructure, and cells are viably sustained within the scaffold.

Optionally the hydrogel comprises protein ligands, e.g. protein ligands involved in cell growth, including, without limitation, growth factors, chemokines, cytokines, fibronectin, cell adhesive peptides (RGDS), laminin, and the like.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Survival of macrophages transplanted into cutaneous wounds. IVIS imaging of macrophage survival in wounds compared to control wounds by luciferase bioluminescence (*p<0.0001 for all timepoints).

FIG. 2. Macrophage transplantation improves cutaneous wound healing. (A) Bar graph showing the difference in time to complete healing between macrophage-treated and control-treated wounds in wild type mice (*p=0.0001). (B) Scar size as a percentage of original wound size in macrophage-treated as compared to control-treated wounds (p=0.237).

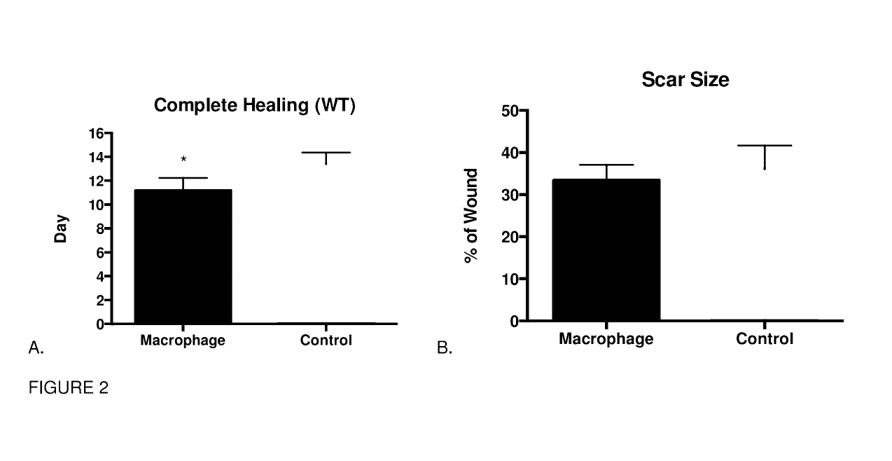

FIG. 3. Macrophage transplantation improves diabetic wound healing. (A) Wound healing curve showing wound size as a percentage of original wound size graphed against time in days (*p<0.01 for all timepoints) in macrophage-treated vs. control-treated diabetic wounds. (B) Scar size as a percentage of original wound size in macrophage-treated as compared to control-treated diabetic wounds (p=0.2851).

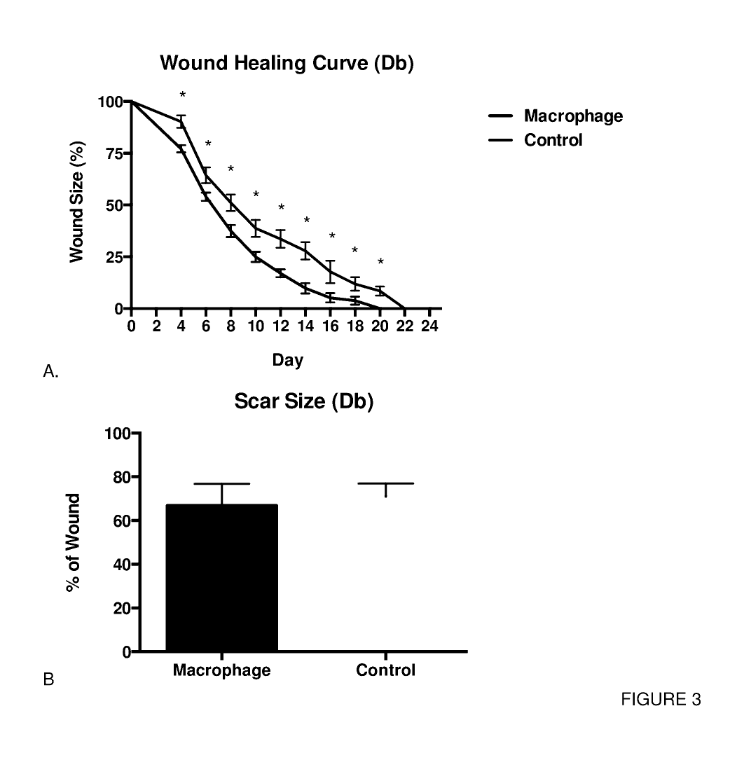

FIG. 4. Single cell transcriptional analysis of transplanted macrophages isolated at day 0, 1, 4, and 7 post-wounding. Hierarchical clustering of macrophages on day 0, 1, 4, and 7 after wound application. Gene expression is presented as fold change from median on a color scale from yellow (high expression, 32-fold above median) to blue (low expression, 32-fold below median), with gray indicating no expression.

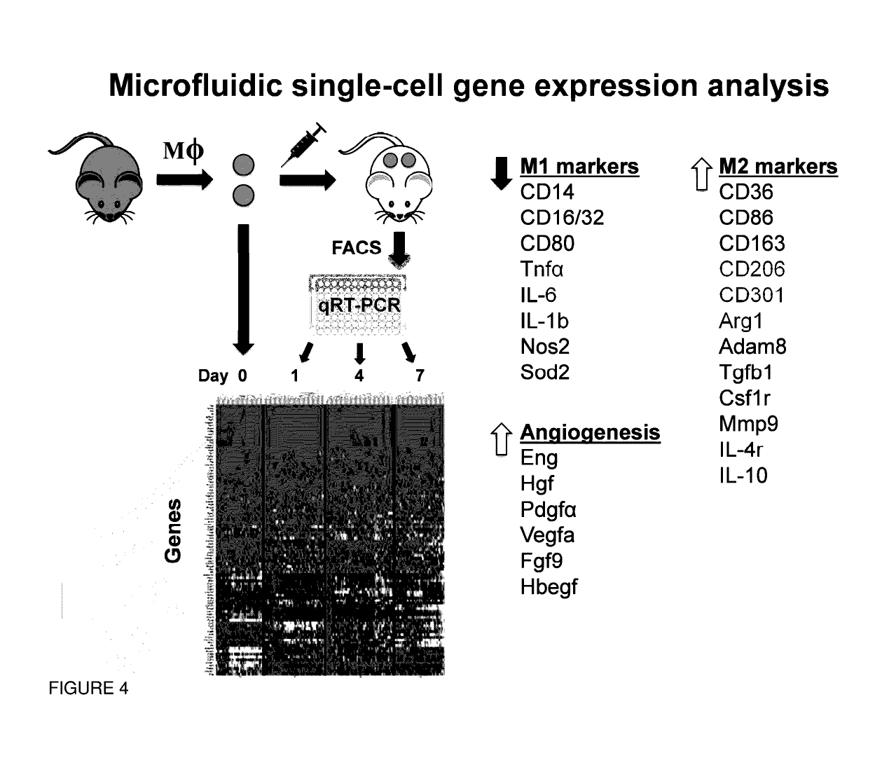

FIG. 5. Monocyte transplantation improves cutaneous wound healing. (A) Wound healing curve showing wound size as a percentage of original wound size graphed against time in days (*p<0.01 for all timepoints) in monocyte-treated and control-treated wounds in Foxn1^{nu} mice. (B) Scar size as a percentage of original wound size in monocyte-treated as compared to control-treated wounds (*p=0.0029).

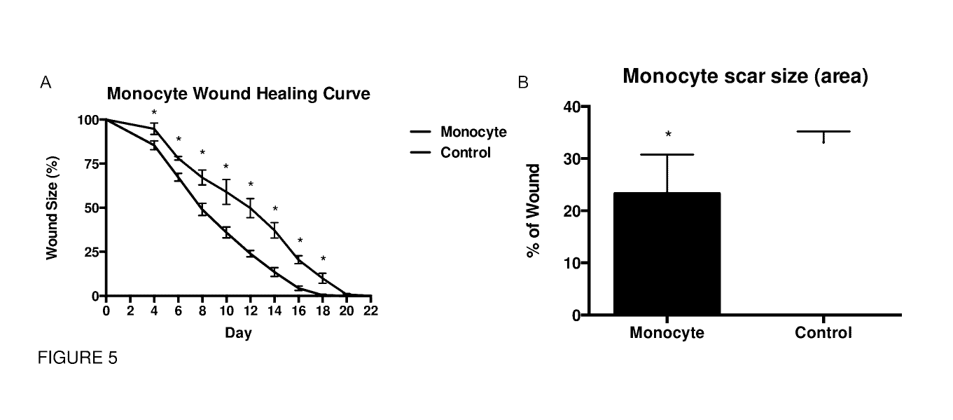

### DESCRIPTION OF THE SPECIFIC EMBODIMENTS

Compositions and methods are provided for wound healing comprising contacting a wound on an individual with a scaffold or matrix, e.g. a hydrogel comprising an effective dose of macrophages or monocyte progenitors thereof. The macrophages may be activated or differentiated from monocytes in vitro, or monocyte progenitor cells can be provided in a scaffold or matrix and allowed to differentiate in situ at the wound site. In some embodiments the scaffold or matrix is a hydrogel is a cross-linked pullulan hydrogel.

The dressings of the invention are suitable for burn patients, diabetic ulcers, venous ulcers, partial- and full-thickness wounds, pressure ulcers, chronic vascular ulcers, trauma wounds, draining wounds, and surgical wounds. The dressing is easily applied in the first few hours following injury and debridement, and can remain in place until wound healing is complete.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Scaffold or matrix. A support that provides for maintaining viability and location of cells when placed over a cutaneous wound.

Hydrogel. Carbohydrate-based hydrogels useful in the methods of the invention maintain viability of entrapped cells for a period of time sufficient to enhance wound healing. Hydrogels are known and used in the art for wound healing. Typically hydrogels are, by weight, up to about 50%, up to about 55%, up to about 60%, up to about 65%, up to about 70%, up to about 75%, up to about 80%, up to about 85%, up to about 90% water, with the remaining weight comprising a suitable polymer, e.g. pullulan and collagen, glycosaminoglycan, acrylate, 2-hydroxymethyl methacrylate and ethylenedimethacrylate copolymer, carboxymethylcellulose, chitosan, gelatin, etc., or other suitable hydrophilic polymers as known in the art. Hydrogels can swell extensively without changing their gelatinous structure and are available for use as amorphous (without shape) gels and in various types of application systems, e.g. flat sheet hydrogels and non-woven dressings impregnated with amorphous hydrogel solution. Flat sheet (film) hydrogel dressings have a stable cross-linked macrostructure and therefore retain their physical form as they absorb fluid.

In some embodiments a cross-linked hydrogel film is fabricated using pullulan and collagen under conditions that provided for cross-linking and pore formation. Collagen is added to a mixture of pullulan, cross-linking agent and pore-forming agent (porogen), where the collagen is provided at a concentration of at least about 1%, and not more than about 12.5% relative to the dry weight of the pullulan. Collagen may be provided at a concentration of about 1%, about 2.5%, about 5%, about 7.5%, about 10%, usually at a concentration of from about 2.5% to about 10%, and may be from about 4% to about 6% relative to the dry weight of the pullulan. The collagen is typically a fibrous collagen, e.g. Type I, II, Ill, etc. Cross-linking agents of interest include sodium trimetaphosphate (STMP) or a combination of or a combination of sodium trimetaphosphate and sodium tripolyphosphate (STMP/STPP). The cross-linking agent can be included in a wt/wt ratio relative to the pullulan of from about 5:1 to about 1:5, and may be about 4:1, 3:1, 2:1, 1.75:1, 1.5:1, 1.25:1, 1:1, 1:1.25, 1:1.5, 1:1.75, 2:1, 3:1, 4:1, etc. Porogens of interest for in-gel crystallization include any suitable salt, e.g. KCl. The porogen can be included in a wt/wt ratio relative to the pullulan of from about 5:1 to about 1:5, and may be about 4:1, 3:1, 2:1, 1.75:1, 1.5:1, 1.25:1, 1:1, 1:1.25, 1:1.5, 1:1.75, 2:1, 3:1, 4:1, etc. The suspension of collagen, pullulan, cross-linker and porogen, in the absence of cells, is poured and compressed to form sheets. Preferred thickness is at least about 1 mm and not more than about 5 mm, usually not more than about 3 mm, and may be from about 1 to 2.5 mm, e.g. about 1.25, 1.5, 1.75, 2 mm thick. Pores are formed in the hydrogel through rapid dessication of swollen hydrogels by phase inversion. Dehydration results in localized super-saturation and crystallization of the porogen. Pullulan and collagen are forced to organize around the crystals in an interconnected network, which results in reticular scaffold formation following KCl dissolution.

The films may be stored in a dried state, and are readily rehydrated in any suitable aqueous medium. The aqueous nature of hydrogel substrates provides an ideal environment for cellular growth and sustainability.

Mechanical features of the hydrogel include average pore size and scaffold porosity. Both variables vary with the concentration of collagen that is present in the hydrogel. For a hydrogel comprising 5% collagen, the average pore size will usually range from about 25 μm to about 50 μm, from about 30 μm to about 40 μm, and may be about 35 μm. For a hydrogel comprising 10% collagen the average pore size will usually range from about 10 μm to about 25 μm, from about 12 μm to about 18 μm, and may be about 15 μm. One of skill in the art will readily determine suitable hydrogels at other collagen concentrations. The scaffold porosity will usually range from about 50% to about 85%, and may range from about 70% to about 75%, and will decrease with increasing concentrations of collagen. Hydrogels lacking collagen do not display any birefringence with polarizing light microscopy, while the hydrogels comprising collagen are diffusely birefringent.

Pullulan. A polysaccharide produced by the fungus Aureobasidium pullulans. It is a linear homopolysaccharide consisting of alpha-(1-6) linked maltotriose units and exhibits water retention capabilities in a hydrogel state which makes it an ideal therapeutic vehicle for both cells and biomolecules. Additionally, pullulan contains multiple functional groups that permit cross-linking and delivery of genetic material and therapeutic cytokines. Furthermore, pullulan-based scaffolds have been shown to enhance both endothelial cell and smooth muscle cell behavior in vitro.

Collagen. As used herein the term “collagen” refers to compositions in which at least about 50%, at least about 60%, at least about 70%, at least about 80%, at least about 90%, at least about 95% or more of the protein present is collagen in a triple helical configuration. Collagens are widely found in vertebrate species, and have been sequenced for many different species. Due to the high degree of sequence similarity between species, collagen from different species can be used for biomedical purposes, e.g. between mammalian species. Typical commercial animal sources include the bovine Achilles tendon, calfskin and the bones of cattle. In some embodiments the collagen used in the preparation of the oriented thin film is Type I, Type II, or Type III collagen, and is derived from any convenient source, e.g. bovine, porcine, etc., usually a mammalian source.

Collagen has a triple-stranded rope-like coiled structure. The major collagen of skin, tendon, and bone is collagen I, containing 2 alpha-1 polypeptide chains and 1 alpha-2 chain. The collagen of cartilage contains only 1 type of polypeptide chain, alpha-1. The fetus also contains collagen of distinctive structure. The genes for types I, II, and III collagens, the interstitial collagens, exhibit an unusual and characteristic structure of a large number of relatively small exons (54 and 108 bp) at evolutionarily conserved positions along the length of the triple helical gly-X-Y portion.

Types of collagen include I (COL1A1, COL1A2); II (COL2A1); III (COL3A1); IV (COL4A1, COL4A2, COL4A3, COL4A4, COL4A5, COL4A6); V (COL5A1, COL5A2, COL5A3); VI (COL6A1, COL6A2, COL6A3); VII (COL7A1); VIII (COL8A1, COL8A2); IX (COL9A1, COL9A2, COL9A3); X (COL10A1); XI (COL11A1, COL11A2); XII (COL12A1); XIII (COL13A1); XIV (COL14A1); XV (COL15A1); XVI (COL16A1); XVII (COL17A1); XVIII (COL18A1); XIX (COL19A1); XX (COL20A1); XXI (COL21A1); XXII (COL22A1); XXIII (COL23A1); XXIV (COL24A1); XXV (COL25A1); XXVII (COL27A1); XXVIII (COL28A1). It will be understood by one of skill in the art that other collagens, including mammalian collagens, e.g. bovine, porcine, equine, etc. collagen, are equally suitable for the methods of the invention.

Supports. A variety of solid supports or substrates may be used with the scaffold or matrix, e.g. a hydrogel, including deformable supports or barriers. By deformable is meant that the support is capable of being damaged by contact with a rigid instrument. Examples of deformable solid supports include polyacrylamide, nylon, nitrocellulose, polypropylene, polyester films, such as polyethylene terephthalate; PDMS (polydimethylsiloxane); tagamet; etc. as known in the art for the fabrication of wound dressings.

Cells. The films of the invention provide a substrate for maintenance of macrophages or monocyte progenitors thereof, which are typically mammalian cells, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human. The cells which are employed may be fresh, frozen, or have been subject to prior culture. The cells may be autologous or allogeneic relative to the intended recipient. Cells can be obtained from a blood draw, e.g. allogeneic or autologous, or may be differentiated in vitro from a stem or progenitor cell population, which include without limitation induced pluripotent stem cells, embryonic stem cells, hematopoietic stem cells, and the like.

In practicing the subject methods, a cell composition that is enriched for macrophages and/or monocyte precursors thereof is administered to an individual in a hydrogel of the invention for the improvement of cutaneous wound healing. In some embodiments the cells are selected for expression of CD14.

By “macrophages”, it is meant a type of leukocyte of the monocyte lineage. Macrophages or the monocyte precursors thereof which are suitable for use in the subject methods can be readily identified by the expression—or in some instances, absence of expression—of one or more of the following marker proteins: CD14, CD86, HLA-DR, MHC-II, CD80, CD40, CD11b, CD11c, F4/80, CD16, CD64, TLR2, TLR4, CD163, and/or CD274 (see, e.g., the working examples herein and Hutchinson et al., J Immunol. 2011 Sep. 1; 187(5):2072-8 and Brem-Exner et al., J Immunol. 2008 Jan. 1; 180(1):335-49). CD14 is a specific marker of both monocytes and macrophages, and can be used as a sole marker for purification of human monocytes from a peripheral blood sample. Other markers include, without limitation, CD206, CD163, SIRPα, CD11b, CD11c, CD36, CD45, CD9, CD32, CD64, CD14, CD166, CD131, etc.

By an enriched cell composition of macrophages or monocytes, it is meant that at least about 50%, about 60%, about 70%, about 80%, about 90%, about 95% or more of the cells of the cell composition are macrophages or monocytes. In some instances, the enriched composition will be a substantially pure population of macrophages or monocytes, whereby “substantially pure” it is meant at least 90% or more of the composition will be of the selected phenotype, e.g. 95%, 98%, and up to 100% of the population.

It will be understood by those of skill in the art that expression levels reflect detectable amounts of the marker (e.g., protein or nucleic acid) on and/or in the cell. A cell that is negative for staining (e.g., the level of binding of a marker specific reagent is not detectably different from a matched control) may still express minor amounts of the marker. And while it is commonplace in the art to refer to cells as “high”, “+”, “positive”, “low”, “−”, or “negative” for a particular marker, actual expression levels are quantitative traits. For example, number of detected molecules can vary by several logs, yet still be characterized as “positive”. When a protein marker is used, the staining intensity (e.g., of a marker-specific antibody) can be monitored by any method suitable for assaying protein expression, e.g., flow cytometry, Western blotting, mass spectrometry, and enzyme-linked immunosorbent assay (ELISA).

As one example, in flow cytometry, lasers detect the quantitative levels of fluorochrome (which is proportional to the amount of cell marker bound by specific reagents, e.g. antibodies). Flow cytometry, or FACS, can also be used to separate cell populations based on the intensity of binding to a specific reagent (or combination of reagents), as well as other parameters such as cell size and light scatter. Although the absolute level of staining may differ with a particular fluorochrome and reagent preparation, the data can be normalized to a control. As such, a population of cells can be enriched for macrophages by using flow cytometry to sort and collect those cells (e.g., only those cells) with the desired profile.

In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining brighter than that of an isotype matched control, but is not as intense as the most brightly staining cells normally found in the population. An alternative control may utilize a substrate having a defined density of marker on its surface, for example a fabricated bead or cell line, which provides the positive control for intensity.

Cell compositions that are enriched for macrophages or monocytes that find use in the subject methods include compositions comprising macrophages that have been acutely isolated from an individual, e.g., the individual undergoing treatment, or a donor individual. Alternatively and more commonly, the subject cell compositions may be prepared in vitro by isolating a population of leukocytes comprising monocytes from an individual, e.g., the individual undergoing treatment, or a donor individual, and culturing the population in vitro to produce a cell composition that is enriched for macrophages. By monocytes it is meant a type of leukocyte (white blood cell) that is part of the innate immune system of vertebrates.

Monocytes have bean-shaped nuclei and constitute 2-10% of all leukocytes in the human body. Monocytes are part of the myeloid lineage, and can act as precursor cells that replenish macrophages and/or dendritic cells under normal states. In response to inflammation, monocytes can move quickly to sites of infection and divide/differentiate into macrophages and/or dendritic cells (e.g., to elicit an immune response). Monocytes can be identified by their large kidney shaped or notched nucleus, as well as by the expression of certain cell surface markers including, for example, CD14.

In some instances, the subject cell compositions that are enriched for macrophages or monocytes are prepared from a heterogeneous population of leukocytes comprising monocytes, that is, a population in which about 60% or less, e.g. 50% or less, 40% or less, 30% or less, 20% or less, or 10% or less of the cells are monocytes. In other instances, the subject cell compositions may be prepared from an enriched population of monocytes, e.g. a population of leukocytes in which about 60% or more, e.g. 70% or more, 75% or more, 80% or more, 85% or more, 90% or more, 95% or more, 98% or more, in some instances substantially all of the cells are monocytes. Cell populations comprising monocytes may be obtained by any convenient method. For example, the monocytes may be obtained from blood (e.g., heparinized blood), bone marrow, and/or spleen tissues. The monocytes may be obtained from peripheral blood mononuclear cells (PBMCs) by Ficoll density gradient separation (e.g., leukopheresis followed by Ficoll density gradient separation). Alternatively, a stem or progenitor cell population can be induced to a monocyte or macrophage phenotype. In some instances, affinity reagents, e.g. antibodies specific for monocyte cell-surface markers, e.g. CD14, may be employed.

In some instances, the donor of the macrophages or of the population of leukocytes comprising monocytes from which the subject cell compositions will be prepared is the same as the individual (the “recipient”) receiving the subject treatment (i.e., the individual suffering from a wound). In other words, the macrophages (or monocytes to be induced to differentiation into macrophages) are drawn from an individual as a blood draw, and the macrophages (or macrophages induced from monocytes) are transferred back (restored) into the same individual. In such an instance, the macrophages or monocytes to be induced to differentiation into macrophages are autologous to the recipient.

In other instances, the donor of the macrophages or of the population of leukocytes comprising monocytes from which the subject cell compositions will be prepared is different from the individual (the “recipient”) receiving the subject treatment. In other words, the macrophages or monocytes to be induced to differentiation into macrophages are allogeneic to the recipient. In such instances, the macrophages or monocytes to be induced to differentiation into macrophages are selected based upon the blood type of the donor and the blood type of the recipient. By blood type, it is meant the presence or absence of A and B antigens and Rh antigen on the donor and recipient's red blood cells. For example, as is well understood in the art, an individual may have neither A or B antigens on his red blood cells (and hence will have antibodies specific for both A and B antigens in his plasma), in which case the individual is “type O”. The individual may have A antigen and not B antigen on his red blood cells (and hence will have antibodies specific for B antigen but not A antigen in his plasma), in which case the individual is “type A.” The individual may have B antigen and not A antigen on his red blood cells (and hence antibodies specific for A antigen but not B antigen in his plasma), in which case the individual is “type B.” The individual may have both A and B antigens on his red blood cells (and hence no antibodies for either A or B antigen in his plasma), in which case the individual is “type AB.” As well known in the art, safe transfusion of donor blood to a recipient can occur if the donor is type O and the recipient is any type; if the donor is type A and the recipient is type A or type AB; if the donor is type B and the recipient is type B or type AB; or if the donor is type AB and the recipient is type AB. Additionally, as is known in the art, the Rh antigen may or may not be present, i.e., the individual is Rh-positive or Rh-negative, respectively. As is well known in the art, safe transfusion of donor leukocytes to a recipient can occur if the donor is type Rh+ or Rh+ and the recipient is type Rh+; or if the donor is type Rh− and the recipient is type Rh−.

Any convenient method for activating or differentiating macrophages in vitro, e.g. as known in the art may be used to produce the subject cell compositions. For example, monocytes may be induced to differentiate into macrophages by culturing in medium containing macrophage colony-stimulating factor (M-CSF); optionally in combination with and interferon gamma (IFN-γ); culturing in medium containing GM-CSF; and/or culturing in medium containing human serum, e.g. from about 7.5% to about 12.5% human serum and may be about 10% human serum. In other words, macrophages can be derived from monocytes when monocytes are contacted with any one of M-CSF, GM-CSF, human serum, M-CSF and γ-IFN, etc. In some embodiments, the subject methods include preparing a cell population that is enriched for macrophages. In such embodiments, any convenient method of producing a cell population enriched for macrophages can be employed.

For example, monocytes (e.g., plastic adherent monocytes) may be cultured in the presence of M-CSF, GM-CSF, 10% human serum, etc., e.g., in a basal medium such as RPMI 1640, for a period of time in a range of from 3 days to 10 days, e.g., 3 days to 9 days, 3 days to 8 days, 3 days to 7 days, 3 days to 6 days, 4 days to 10 days, 4 days to 9 days, 4 days to 8 days, 4 days to 7 days, 4 days to 6 days, 4.5 days to 5.5 days, 5 days to 10 days, 5 days to 9 days, 5 days to 8 days, 5 days to 7 days, 5 days to 6 days, 6 days to 10 days, 6 days to 9 days, 6 days to 8 days, 6 days to 7 days, 7 days to 10 days, 7 days to 9 days, 7 days to 8 days, 3 days, 4 days, 5 days, 6 days, 7 days, 8 days, 9 days, or 10 days. Any convenient M-CSF or GM-CSF can be used. Suitable examples of M-CSF include, but are not limited to: recombinant M-CSF; recombinant human M-CSF; recombinant mouse M-CSF; recombinant rat M-CSF; and the like. In some embodiments, monocytes are contacted with M-CSF at a concentration in a range of from 2 ng/ml to 8 ng/ml, e.g., 2.5 ng/ml to 7.5 ng/ml, 3 ng/ml to 7 ng/ml, 3.5 ng/ml to 6.5 ng/ml, 4 ng/ml to 6 ng/ml, 4.5 ng/ml to 5.5 ng/ml, or 5 ng/ml. In some cases, the M-CSF can be carried on serum albumin, e.g., human serum albumin, e.g., 0.1% human serum albumin.

In some cases, monocytes are cultured in the presence of M-CSF in a basal medium, e.g., an RPMI 1640-based medium. In some cases, monocytes are cultured in the presence of M-CSF in a basal medium (e.g., an RPMI 1640-based medium) that does not include phenol red. In some cases, the medium further includes one or more of serum (e.g., 10% human AB serum); L-glutamine (e.g., 2 mM L-glutamine); penicillin (e.g., 100 U/ml); and streptomycin (e.g., 10 mg/ml).

Alternatively macrophages and/or monocytes can be activated ex vivo by culture in the presence of one or more of IFN-γ, IFN-α, IFN-β, GM-CSF, IL-4, IL-10, IL-13, IL-17, IL-1β, TNFα, CCL2, MIP1a, MIP1b, CCL22, IL-8, LPS, muramyl dipeptide, etc.

The cells can be plated at any convenient density. In some cases, the cells are cultured at a density in a range of from 1×10⁵ to 1×10⁹ monocytes per 175 cm² (e.g., 5×10⁵ to 5×10⁸ monocytes per 175 cm²; 1×10⁶ to 2×10⁸ monocytes per 175 cm²; 5×10⁶ to 1×10⁸ monocytes per 175 cm²; 8×10⁶ to 6×10⁷ monocytes per 175 cm²; 1×10⁷ to 6×10⁷ monocytes per 175 cm²; 2×10⁷ to 5×10⁷ monocytes per 175 cm²; or 2.5×10⁷ to 4×10⁷ monocytes per 175 cm²).

In some cases, cultures are gently washed to select for adherent cells and fresh medium can then be added to the adherent cell layer. For example, in some cases, after monocytes have been contacted with M-CSF for a period of time in a range of from 12 hours to 36 hours (e.g., 18 hours to 30 hours, 20 hours to 28 hours, 22 hours to 26 hours, or 24 hours) cultures can be gently washed to select for adherent cells and fresh medium can then be added to the adherent cell layer. Such washes and selection can be repeated at any convenient interval thereafter (e.g., every 12 hours, every 24 hours, every 36 hours, every 48 hours, and the like).

In some cases, adherent cells are harvested (e.g., with a cell scraper, using trypsin-EDTA treatment, and the like) and washed (e.g. in phosphate-buffered saline (PBS); physiological saline solution, e.g., containing 5% human albumin for infusion; and the like) before use. Macrophages for administration to human individuals can be prepared under strict Good Manufacturing Practice (GMP) conditions.

Other standard cell culture components that are suitable for inclusion in the culturing process include, but are not limited to, a vitamin; an amino acid (e.g., an essential amino acid); a pH buffering agent; a salt; an antimicrobial agent (e.g., an antibacterial agent, an antimycotic agent, etc.); serum; an energy source (e.g., a sugar); a nucleoside; a lipid; trace metals; a cytokine, a growth factor, a stimulatory factor, and the like. Any convenient cell culture media can be used. Various cell types grow better in particular media preparations. Accordingly, any convenient cell culture media can be used and may be tailored to macrophage/monocyte cell culture.

In some embodiments, a subject cell culture medium (e.g., a basal culture medium) includes animal serum (e.g., fetal bovine serum (FBS); fetal calf serum (FCS), bovine serum, chicken serum, newborn calf serum, rabbit serum, goat serum, normal goat serum (NGS); horse serum; lamb serum, porcine serum, human serum (e.g., human AB serum, AB-human serum, and the like). A wide range of serum concentrations can be used. A cell culture composition of the present disclosure can have a concentration of serum in a range of from 1% to 50% (e.g., from 2% to 40%, from 2% to 30%, from 2% to 25%, from 2% to 20%, from 2% to 15%, from 2% to 10%, from 2% to 7%, from 2% to 5%, from 3% to 12%, from 5% to 15%, from 8% to 12%, from 9% to 11%, from 8% to 20%, 5%, 6%, 7%, 8%, 9%, 10%, 11%, 12%, 13%, 14%, or 15%). In some embodiments, a subject cell culture medium is serum free.

The population of macrophages or monocytes so prepared will be an enriched population of macrophages or monocytes. In other words, at least about 50%, about 60%, about 70%, about 80%, about 90% about 95% of the cells of the population are of the selected phenotype. In some instances, the enriched population will be a substantially pure population of macrophages or monocytes, whereby “substantially pure” it is meant at least 95% or more of the population be of the selected phenotype, e.g. 95%, 98%, and up to 100% of the population.

In some instances, it may be advantageous to enrich for (i.e., purify) the macrophages or monocytes. “Enriching” refers to a step in which the fraction (i.e., percentage) of macrophages or monocytes present in the final cell population is greater than the fraction of macrophages or monocytes present in the starting cell population. For example, if an isolated population of macrophages is to be employed, it may be necessary to mechanically enrich the population for macrophages. As another example, when culturally enriched macrophages are to be employed (e.g., macrophages prepared by culturing monocytes ex vivo), it may be desirable to further enrich the population of macrophages. By “mechanical enriching” it is meant a mechanical separation of cells of interest (e.g., macrophages or monocytes) from a cell population, for example by positive selection of the cells of interest or by negative selection (depletion) of the cells not of interest. Examples of mechanical enrichment strategies include, but are not limited to: cell sorting using flow cytometry (e.g., fluorescence activated cell sorting (FACS)), cell sorting using magnetic bead sorting (e.g., magnetic beads conjugated to antibodies and/or ligands that bind to monocyte or macrophage markers), immunopanning (e.g., using a solid support conjugated to antibodies and/or ligands that bind to monocyte or macrophage markers), adherence to plates (for macrophages), and the like. Macrophages may be selected or enriched by using a screenable or selectable reporter expression cassette comprising an lineage-specific transcriptional regulatory element operably linked to a reporter gene.

In some cases, multiple types of enriching can be used. In some cases, enriching can happen in more than one step. For example, in some cases, monocytes are cultured for a period of time in the presence of M-CSF. A step of enrichment can be performed at any point in the process. For example, cells can be mechanically sorted (e.g., the cell population can be enriched for macrophages, e.g. using affinity reagents specific for macrophages or monocytes, e.g. CD14-specific antibodies or a CD14 affinity column) prior to culture in the presence of M-CSF or at point after culture has commenced. In some cases, after monocytes are cultured in the presence of M-CSF the resulting cell population is then enriched for macrophages (e.g., cells of the cell population are subjected to flow cytometry) to further enrich the cell population for macrophages.

Enrichment using antibodies (e.g., magnetic cell sorting, FACS, and the like) specific for cell surface markers of macrophages or monocytes have the advantage of not requiring genetic modification of the cells to be enriched. Magnetic cell sorting and FACS have the ability to analyze multiple surface markers simultaneously, and they can be used to sort macrophages or monocytes based on the expression levels of cell surface markers.

Once produced, the presence and/or percent of macrophages in the population can be readily verified by, for example, detecting the expression of the one or more proteins, e.g. CD14, CD86, HLA-DR, MHC-II, CD80, CD40, CD11b, CD11c, F4/80, CD16, CD64, TLR2, TLR4, CD163, and/or CD274. In some embodiments, the subject methods include the step of verifying the presence of macrophages or monocytes in a cell population, e.g., after culturing as descried above, by detecting the expression of one or more of CD14, CD206, CD163, SIRPα, CD11b, CD11c, CD36, CD45, CD9, CD32, CD64, CD14, CD166, CD131, which are positively expressed by macrophages. In some embodiments, the subject methods include the step of verifying the presence of macrophages in a cell population, e.g., after culturing as described above, by detecting the expression of one or more of markers that are indicative of macrophages.

Additionally or alternatively, verifying can rely on cellular phenotypes, e.g., gene or protein expression, drug metabolism profile, responsiveness to particular drugs, etc., that are characteristic of macrophages or monocytes. Marker expression (e.g., as determined by measuring protein and/or RNA levels) may be examined before, during, and/or after the production of macrophages by the subject methods. The expressed set of markers may be compared against other subsets of cells (e.g., untreated precursor monocyte cells). In some cases, cells of a subject population are assayed in order to measure the percent of cells in the population that are macrophages. In some cases, a cell population is enriched for macrophages, which increases the percent of cells of a cell population that are macrophages. In some cases, enriching occurs simultaneously verifying the presence of the macrophages (e.g., when using flow cytometry to enrich a cell population for macrophages).

In some embodiments, verifying includes contacting cells of a cell population with specific binding agents (e.g., an antibodies, nucleic acid probes, etc.) that are specific for macrophage or monocyte markers (e.g., protein, mRNA etc.) and determining the percentage of cells of the cell population that are macrophages or monocytes. Suitable markers are listed above. Verification of the presence of macrophages or monocytes can be performed at any point in the process of producing macrophages and seeding of the hydrogel. For example, the percent of Macrophages can be determined on any day during culture in the presence of M-CSF, any day during culture in the presence of IFN-gamma, after culturing cells in the presence of IFN-gamma, and/or after a step of enrichment (e.g., mechanical enrichment).

Cells may be initially seeded or grown for a period of from about 1 hour, about 6 hours, about 12 hours, about 18 hours, about 24 hours, about 36 hours, to about 48 hours on the film in vitro, where the film may be placed in a suitable vessel for culture, e.g. a flask, plate, multiwell plate, etc. Alternatively the cells can be seeded onto the film immediately prior to use. The cells are grown in vitro in an appropriate liquid nutrient medium. Any cell culture medium appropriate for growth and differentiation of cells may be used. These include, but are not limited to, DMEM, MEM, M-199 and RPMI. Supplements, as are known in the art, may be added to the culture medium and include serum (e.g., FBS or calf serum), serum-containing supplements (NU-SERUM), and serum-free supplements (MITO+).

Generally, the seeding level will be at least about 10 cells/ml, more usually at least about 100 cells/ml and generally not more than about 10⁶ cells/ml, usually not more than about 10⁵ cells/ml. The number of cells on the film may be from about 10⁴ cells/cm² of film, up to about 10⁸ cells/cm² of hydrogel, and may be present at a concentration of from about 10⁵ to about 10⁷ cells/cm² of film, assuming that the film is a planar configuration of from about 0.5 to about 2.5 mm in thickness.

Regenerative factors. Polypeptide growth factors and cell-signaling molecules may be included in a hydrogel film. Protein ligands are printed on the hydrogels by precise micro-contact printing methods. Alternatively the proteins may be included in the initial fabrication of the matrix. Polypeptides of interest as growth factors include, without limitation, the following molecules, where one or more of the factors may be patterned on a matrix. The native form of the polypeptides may be used, or variants thereof, e.g. truncated versions that maintain biological activity; stabilized variants; conjugated engineered for improved adhesion to the hydrogel matrix, and the like.

Platelet-derived growth factor (PDGF) is a family of potent activators for cells of mesenchymal origin, and a stimulator of chemotaxis, proliferation and new gene expression in monocytes, macrophages and fibroblasts, accelerating ECM deposition. This family of growth factors exists in both homo- and heterodimeric forms.

Cytokines of the transforming growth factor-β family (TGF-β) are multifunctional regulators of cell growth, differentiation and ECM formation. In mammals, there are three isoforms, TGF-β1, TGF-β2 and TGF-β3. In particular, in relation to wound healing in the skin, TGF-β1 and TGF-β2 are implicated in cutaneous scarring, whereas TGF-β3 is known to have an anti-scarring effect.

Bone morphogenetic proteins (BMPs) are members of the TGF-β superfamily. There are 15 members and although they are known for their role in bone and cartilage formation, they have diverse roles in many other developmental processes.

Fibroblast growth factors (FGFs) are a family of 21 isoforms with a broad spectrum of activities, including regulation of cell proliferation, differentiation and migration. FGFs 1, 2, 5, 7 and 10 are upregulated during adult cutaneous wound healing. bFGF may have the ability to accelerate tissue regeneration in artificial dermis.

Vascular endothelial growth factor (VEGF) is induced during the initial phase of skin grafting, where endogenous fibrin clots are known to form a provisional matrix and to promote angiogenesis. Growth factors such as VEGF increase in such wounds to stimulate angiogenesis.

Epidermal growth factor (EGF) has been implicated in wound healing and homeostasis in a number of tissues.

Hepatocyte growth factor/scatter factor (HGF/SF) is a pleiotrophic growth factor produced principally by cells of mesenchymal origin. HGF has been implicated in enhancing the cutaneous wound healing processes of re-epithelialization, neovascularization and granulation tissue formation.

Antimicrobial agents. The hydrogels may further comprise antimicrobial agents. Agents of interest include a wide variety of antibiotics, as known in the art. Classes of antibiotics include penicillins, e.g. penicillin G, penicillin V, methicillin, oxacillin, carbenicillin, nafcillin, ampicillin, etc.; penicillins in combination with β lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Antiviral agents, e.g. acyclovir, gancyclovir, etc. may also be included.

Wound dressing. films of the invention find use as a wound dressing, or artificial skin, by providing an improved substrate that minimizes scarring. An effective bioactive wound dressing can facilitate the repair of wounds that may require restoration of both the epidermis and dermis. For example, a hydrogel thin film is placed onto, and accepted by, the debrided wound of the recipient and provide a means for the permanent re-establishment of the dermal and epidermal components of skin. The graft suppresses the formation of granulation tissue which causes scarring.

Additional criteria for biologically active wound dressings include: rapid adherence to the wound soon after placement; proper vapor transmission to control evaporative fluid loss from the wound and to avoid the collection of exudate between the wound and the dressing material. Skin substitutes should act as barrier to microorganisms, limit the growth of microorganisms already present in the wound, be flexible, durable and resistant to tearing. The substitute should exhibit tissue compatibility, that is, it should not provoke inflammation or foreign body reaction in the wound which may lead to the formation of granulation tissue. An inner surface structure of an hydrogel thin film is provided that permits ingrowth of fibro-vascular tissue. An outer surface structure may be provided to minimize fluid transmission and promote epithelialization.

Typical bioabsorbable materials for use in the fabrication of porous wound dressings, skin substitutes and the like, include synthetic bioabsorbable polymers such as polylactic acid or polyglycolic acid, and also, biopolymers such as the structural proteins and polysaccharides. The finished dressing prior to cell seeding is packaged and preferably radiation sterilized. Such biologically active products can be used in many different applications that require the regeneration of dermal tissues, including the repair of injured skin and difficult-to-heal wounds, such as burn wounds, venous stasis ulcers, diabetic ulcers, etc.

### Devices and Methods

Devices are described here for accelerating wound healing and/or ameliorating the formation of scars and/or keloids at a wound site. The scars may be any type of scar, e.g., a normal scar, a hypertrophic scar, etc. In general, the devices are configured to be removably secured to a skin surface near a wound. The devices of the invention comprise a scaffold or matrix, e.g. a hydrogel film seeded with an effective dose of macrophages and/or monocyte progenitors of macrophages. Usually the film is seeded with cells prior to use, e.g. by culturing cells in the hydrogel for about 1 to about 24 hours. The film may optionally comprise regenerative protein factors, as described herein, which protein factors may be specifically patterned on the film, or may be integrated in the film, or otherwise coupled to the film. A diverse array of active agents or ingredients may be present in the compositions, as described above. Depending on the nature of the agent, the amount of active agent present in the composition may ranges from about 0.2 to 10%, e.g., from about 0.2 to 5%, e.g., from about 0.5 to 5%. The pH of the patch compositions typically is one that lies in a physiologically acceptable range, where the pH typically ranges from about 3.0 to 8.0 and more typically ranges from about 4.0 to 7.0.

The wound dressing may be attached or adhered to a substrate, e.g. a breathable protective layer, or other protective layer. Alternatively the cell-containing dressing may be separately configured from a protective dressing. In certain embodiments, a cell-containing dressing composition may be present on a support or backing. The support is generally made of a flexible material which is capable of fitting in the movement of the human body and includes, for example, various non-woven fabrics, woven fabrics, spandex, flannel, or a laminate of these materials with polyethylene film, polyethylene glycol terephthalate film, polyvinyl chloride film, ethylene-vinyl acetate copolymer film, polyurethane film, and the like. By “flexible” it is meant that the support may be substantially bent or folded without breaking, tearing, ripping, etc. The support may be porous or non-porous, but is typically non-porous or impermeable to the hydrogel composition, active agent if employed and fluids, e.g., any fluids exuded from the wound site.

The length and width dimensions of the support are typically substantially commensurate, including exactly commensurate, with the length and width dimensions of the hydrogel patch composition with which it is associated. The support layer typically may have a thickness that ranges from about 10 μm to about 1000 μm, but may be less than about 10 μm and/or greater than 1000 μm in certain embodiments.

In addition to the scaffold or matrix, e.g. hydrogel patch composition and the optional support layer, the subject patches may also include a release film on the surface of the hydrogel composition layer opposite the backing that provides for protection of the hydrogel composition layer from the environment. The release film may be any convenient material, where representative release films include polyesters, such as PET or PP, and the like.

The shape of the dressing may vary, where representative shapes include square, rectangle, oval, circle, triangular, etc. The size of the dressing may also vary, where in many embodiments the size ranges from about 1 cm² or less to about 1000 cm² or more, e.g., in certain embodiments ranges from about 10 to about 300 cm², e.g., from about 20 to about 200 cm², e.g., about 130 cm² to about 150 cm². In certain embodiments, the surface area is sufficient to cover a substantial portion or even the entire truck or even a substantial portion of the entire body or even the entire body of a subject. Accordingly, the surface area may range from about 1000 cm² to about 5000 cm² or more. It should be noted that the above manufacturing protocol is merely representative. Any convenient protocol that is capable of producing the subject compositions, as described above, may be employed.

The subject methods find use in any application in which the treatment of a wound of a subject is desired. Generally, such subjects are “mammals” or “mammalian”, where these terms are used broadly to describe organisms which are within the class mammalia, including the order carnivore (e.g., dogs and cats), rodentia (e.g., mice, guinea pigs, and rats), and primates (e.g., humans, chimpanzees, and monkeys). In many embodiments, the subject is a human.

Accordingly, the subject methods may be used to treat a wide variety of open- and closed-skin wounds such that the subject methods may be used to treat wounds that have resulted from a variety of causes, e.g., as a result of a condition such as a disease state, a physical injury such as a fall, scrape, stab wound, gun shot, surgical wound, infection, etc., wartime injuries such as bombs, bullets, shrapnel. Likewise, the subject methods may treat wounds of various dimensions. For example, the subject methods may be employed to with both deep tissue wounds and shallow or superficial wounds, where certain wounds may have depths that reach the muscle. Wounds may be confined to the epidermis such that they do not penetrate into the dermal layer, may be as deep as the dermis or deeper, e.g., may penetrate to or through the dermis and even to or through the subcutaneous tissue layer or deeper, e.g., may penetrate through or to the muscle layer or further. For example, the subject methods may be used to debride wounds that having a depth that ranges from about 0.005 mm to about 2.35 mm, e.g., from about 0.007 mm to about 2.3 mm, e.g., from about 0.01 mm to about 2 mm.

Types of wounds that may be treated with the subject invention include, but are not limited to, ulcers, including pressure ulcers, diabetic ulcers (e.g., diabetic foot ulcers), venous ulcers, lower leg ulcer, etc.; burns (first, second and third degree burns) including scalds, chemical burns, thermal burns such as flame burns and flash burns, ultraviolet burns, contact burns, radiation burns, electrical burns, etc.; bone infections (osteomyelitis); gangrene; skin tears or lacerations, such as made by knives, etc.; abrasions; punctures such as made by nails, needles, wire, and bullets, etc.; incisions such as made by knives, nails, sharp glass, razors, etc.; avuls; amputations; post-operative infections; surgical wounds; brown recluse spider wounds; failing or compromised skin/muscle grafts or flaps; bites; slash wounds, i.e., a wound where the length is greater than the depth; bruises; and the like, or a combination of one or more of the above.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

Such biologically active products can be used in many different applications that require the regeneration of dermal tissues, including the repair of injured skin and difficult-to-heal wounds, such as burn wounds, venous stasis ulcers, diabetic ulcers, etc.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXPERIMENTAL

### Example 1

Carbohydrate-based hydrogels were fabricated using pullulan (Mₓ 200,000, Hayashibara

Laboratories, Okayama, Japan). Collagen was prepared from rat tail collagen type 1 solution (Sigma-Aldrich, St. Louis, Mo.). Cross-linking was performed with sodium trimetaphosphate (STMP, Sigma-Aldrich) under alkaline conditions with sodium hydroxide (Sigma-Aldrich). Potassium chloride salt (KCl, Sigma-Aldrich) was used as a porogen for in-gel crystallization. 100% ethyl alcohol (Sigma-Aldrich) was used for hydrogel dehydration. Pullulanase (Sigma-Aldrich) was prepared in a concentration of 4U/mL in phosphate buffered saline (PBS) (Gibco, Grand Island, N.Y.). Collagenase A (Roche, Indianapolis, Ind.) was prepared in a concentration of 2 mg/mL in PBS. Methylene blue (Sigma-Aldrich) was used to quantify STMP cross-linking per previously published methods. All aqueous solutions were prepared in deionized water. All compounds and reagents were used without further purification.

Hydrogel fabrication. Based on previously published methods, 2 g of pullulan was mixed with 2 g of STMP and 2 g KCl in 50 mg NaOH dissolved in 10 mL of deionized H₂O. Collagen was then added at a concentration of 0, 5, or 10% of the weight of pullulan. The composite mixture was mechanically stirred for 30 minutes at 4° C. The mixture was then poured onto Teflon sheets and compressed to create from 0.5 to 2.5 mm thick films. Hydrogel films were then dehydrated in 100% ethyl alcohol for 15 minutes and allowed to dry overnight. Dried films were then washed in PBS at room temperature until the wash pH was 7.0 and stored at 4° C. until further use. 6 mm punch biopsy disks of 1 mm thickness were used for all experiments. Films were sterilized overnight under UV light in a cell culture hood prior to experiments.

Mice. Mice were bred and maintained at the Stanford University Research Animal Facility in accordance with Stanford University guidelines. All the animals were housed in sterile micro-insulators and given water and rodent chow ad libitum. FVB/NJ, FVB-Tg(CAG-luc,-GFP)L2G85Chco/J, and FVB.BKS(D)-Lepr^{db}/ChuaJ strains were obtained from Jackson laboratories. A leptin receptor deficient model of diabetes was used as a model for diabetic wound healing. In this model, the mice have long-term hyperglycemia primarily due to severe insulin resistance that persists in the fed state despite continued extreme hyperinsulinemia.

Monocytes were isolated from peripheral blood cells using Leukocyte Reduction System chambers, which are essentially buffy coats, performing Magnetic Activated Cell Sorting (MACS) using selection for CD14+ cells. Typically greater than 95% purity is achieved by this one-step method.

Macrophage Differentiation. Mouse macrophages were generated as previously described. Briefly, bone marrow cells were isolated from FVB-Tg(CAG-luc,-GFP)L2G85Chco/J mice and differentiated in IMDM+ GlutaMax (Life Technologies) supplemented with 10% fetal bovine serum (HyClone), 100 U/mL penicillin and 100 mg/mL streptomycin (Life Technologies), and 10 ng/mL murine M-CSF (Peprotech). Cells were cultured on petri dishes and incubated at 37 degrees C. with 5% carbon dioxide for approximately 7 days, at which point they exhibited morphological changes characteristic of macrophage development. Macrophages were prepared for transplantation by washing plates twice with phosphate buffered saline (PBS), incubating plates for approximately 10 minutes with TrypLE (Life Technologies) at 37 degrees C., then removing cells from plates with cell lifters (Corning). Macrophages were washed and centrifuged to remove excess TrypLE, then counted and diluted to appropriate concentrations for transplantation.

Human macrophages were purified from human monocytes and then cultured for one week in the presence of 10% AB human serum. They were further enriched by selecting for adherent cells.

Wound Healing Model and Assessment. A splinted excisional model of murine wound healing was utilized to minimize wound contracture. Briefly, a 6 mm full-thickness wound is created in duplicate on the shaved dorsum of anesthetized mice. A donut-shaped silicone splint with a 10 mm diameter is centered on the wound and fixed to the skin using an adhesive (Krazy Glue, Elmer's Inc., Columbus, Ohio) and interrupted 6-0 nylon sutures (Ethicon Inc., Somerville, N.J.). A transparent dressing (Tegaderm, 3M, St. Paul, Minn.) is then applied to the wound.

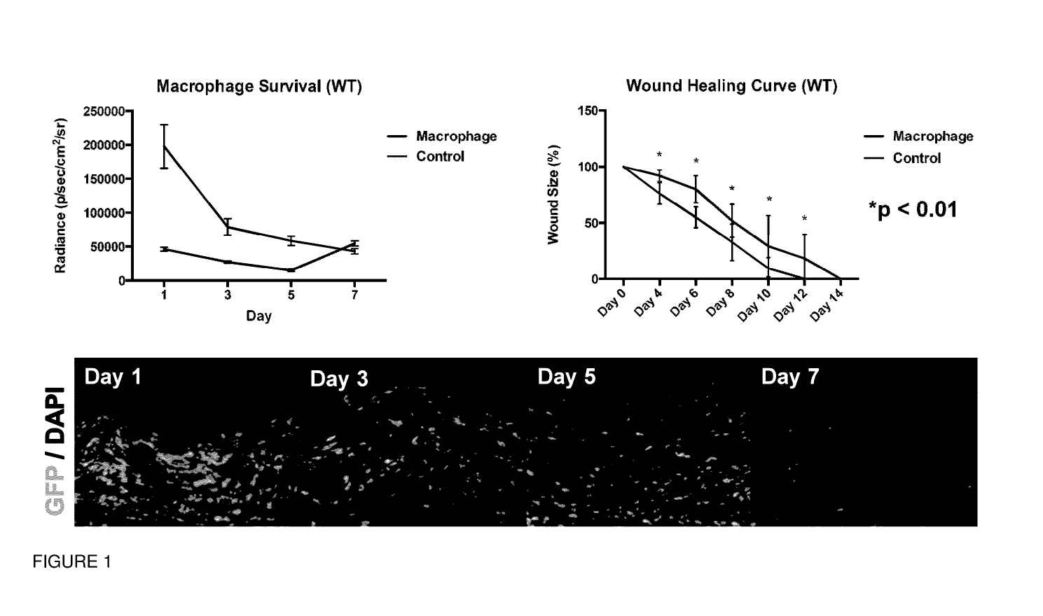

Macrophage Transplantation. After 10 days in culture with M-CSF, adherent cells exhibited the characteristic morphology of macrophage differentiation as previously described (8) (FIG. 1A). Macrophages were then seeded (in PBS) onto pullulan-collagen composite dermal hydrogels and transplanted (2.5×10⁵ cells per wound) into 6-mm full-thickness splinted excisional wounds on the dorsum of FVB/NJ mice (background of donor strain matched to recipient) at the time of wounding (FIG. 1B). At this time, control hydrogels (PBS only; no cells) were also transplanted and wound healing outcomes were assessed. The survival, localization, and behavior of transplanted macrophages in the wound site were characterized using IVIS imaging and histologic analysis of GFP fluorescence. For the experiments with monocytes, monocytes selected as desribed above were seeded onto the hydrogels in the same numbers as macrophages.

Complete healing was defined as the time when the full thickness excisional wounds have completely re-epithelialized on gross examination with macroscopic images.

What is claimed is:

1. A method of enhancing would healing in an individual, comprising:
contacting a cutaneous would with a matrix or scaffold film comprising an effective dose of a cell composition comprising macrophages or monocyte progenitors thereof, where the macrophages or monocyte progenitors thereof are localized to the site of the wound by the matrix or scaffold for a period of time sufficient to enhance healing.

2. The method of claim 1, wherein the matrix or scaffold film is a hydrogel film.

3. The method of claim 2, wherein the individual is a human.

4. The method of claim 2, wherein the hydrogel is a pullulan-collagen composite hydrogel.

5. The method of claim 2, wherein the effective dose of macrophages or monocyte progenitors thereof is from about 10⁴ cells/cm² of hydrogel, up to about 10⁸ cells/cm² of hydrogel film.

6. The method of claim 5, wherein the effective dose of macrophages or monocyte progenitors thereof is from about 10⁵ to about 10⁷ cells/cm² of hydrogel.

7. The method of claim 2, wherein macrophages or monocyte progenitors thereof are selected for expression of CD14.

8. The method of claim 7, wherein the cell composition is at least 50% CD14⁺ cells.

9. The method of claim 8, wherein the CD14+ cells are monocytes.

10. The method of claim 8, wherein the CD14+ cells are macrophages.

11. The method of claim 10, wherein the macrophages are derived by in vitro culture of monocytes in the presence of M-CSF, GM-CSF or human serum.

12. The method of claim 11, wherein the macrophages are further selected by adherence.

13. The method of claim 1, wherein the cell composition is autologous relative to the individual.

14. The method of claim 1, wherein the cell composition is allogeneic relative to the individual.

15. The method of claim 1, wherein the wound is a chronic wound.

16. A hydrogel and cell composition for use in the method of any one of claims 1-15.

17. The composition of claim 16, wherein the hydrogel is a pullulan-collagen hydrogel film with controlled porosity, which cross-linked to form a reticular scaffold.

18. The composition of claim 17, wherein said hydrogel comprises collagen at a concentration of from about 1 to about 12.5%.

19. The composition of claim 18, wherein the hydrogel comprises pores of from about 25 μm to about 50 μm in diameter.

20. The composition of claim 16, further comprising a dressing suitable for wound repair.

21. The composition of claim 20, wherein the dressing comprises a breathable protective layer.
